(19) United States
(12) Patent Application Publication
(10) Pub. No.: US 2009/0149434 A1
(43) Pub. Date: Jun. 11, 2009

(54) FORMULATIONS WITH IMPROVED BIOAVAILABILITY, COMPRISING A STEROID DERIVATIVE AND A POLYGLYCOLYSED GLYCERIDE

(76) Inventor: Joseph Podolski, The Woodlands, TX (US)

Correspondence Address: HOWREY LLP - East, C/O IP DOCKETING DEPARTMENT, 2941 FAIRVIEW PARK DR, SUITE 200, FALLS CHURCH, VA, 22042-2924, US

(21) Appl. No.: 12/088,060

(22) PCT Filed: Sep. 29, 2006
(86) PCT No.: PCT/US06/38637

§ 371 Date: Aug. 11, 2008

Related U.S. Application Data
(60) Provisional application No. 60/721,653, filed on Sep. 29, 2005.

(51) Int. Cl. A61K 31/573; A61P 15/04

(52) U.S. Cl. 514/179

(57) ABSTRACT

Compositions comprising a polyglycolysed glyceride, such as GELUCIRE, and a steroid related compound are provided.

This application claims the benefit, under 35 U.S.C. 119(e), of U.S. Provisional Patent Application No. 60/721,653.

### FIELD OF THE INVENTION

The present invention relates generally to compositions comprising steroids and, in particular, to compositions with potent antiprogestational activity, minimal antiglucocorticoid activity and improved bioavailability, comprising a 19-norprogesterone I derivative and a polyglycolysed glyceride. The present invention also relates to methods using the compositions.

### BACKGROUND OF THE INVENTION

There have been numerous attempts over the past few decades to prepare steroids with antihormonal activity. It has been generally recognized for some years, that antiprogestational steroids would find wide applicability in population control, while antiglucocorticoids would be extremely valuable in the treatment of, for example, Cushing's syndrome and other conditions characterized by excessive endogenous production of cortisone.

For purposes of contraception, it would be advantageous to have compounds which possess antiprogestational activity without (or with minimal) antiglucocorticoid activity. Although there have been a number of attempts to modify the mifepristone structure in order to obtain separation of the antiprogestational activity from the antiglucocorticoid activity, this goal has not yet been fully achieved. As such, there remains a need in the art for the development of new formulations comprising steroids which possess antiprogestational activity with minimal antiglucocorticoid activity.

U.S. Pat. Nos. 6,861,415 and 6,900,193, both incorporated herein by reference, disclose new compounds which possess antiprogestational activity with minimal antiglucorticoid activity. The compounds are steroid derivatives, and more specifically they are structural modifications of 19-norprogesterone I, such as 17-α-substituted-1′-β-substituted-4-aryl and 21-substituted 19-norpregnadienedione, and are poorly soluble in water. Therefore, a need remains in the art to develop formulations comprising the steroid derivatives with increased solubility and improved bioavailability.

### SUMMARY OF THE INVENTION

The present invention provides new formulations with potent antiprogestational activity, minimal antiglucocorticoid activity and improved solubility.

More particularly, the present invention provides compositions comprising GELUCIRE and a compound having the following general formula I:

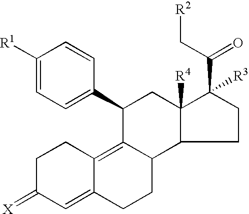

Wherein: R¹ is a functional group including, but not limited to, —OCH₃, —SCH₃, —N(CH₃)₂, —NHCH₃, —NC₄H₈, —NCsHo, —NC₄H₈O, —CHO, —CH(OH)CH₃, C(O)CH₃, —O(CH₂)₂N(CH₃)₂, —O(CH₂)₂NC₄H₈ and —O(CH₂)₂NC₅H₁₀; R² is a functional group including, but not limited to, hydrogen, halogen, alkyl, acyl, hydroxy, alkoxy (e.g., methoxy, ethoxy, vinyloxy, ethynyloxy, cyclopropyloxy, etc.), acyloxy (e.g., formyloxy, acetoxy, propionyloxy, heptanoyloxy, glycinate, etc.), alkylcarbonate, cypionyloxy, S-alkyl, —SCN, S-acyl and —OC(O)R₆, wherein R₆ is a functional group including, but not limited to, alkyl (e.g., methyl, ethyl, etc.), alkoxyalkyl (e.g., —CH₂OCH₃) and alkoxy (—OCH₃); R³ is a functional group including, but not limited to, alkyl (e.g., methyl, methoxymethyl, etc.), hydroxy, alkoxy (e.g., methoxy, ethoxy, methoxyethoxy, vinyloxy, etc.), and acyloxy; R⁴ is a functional group including, but not limited to, hydrogen and alkyl; and X is a functional group including, but not limited to, ═O and ═N—OR⁵, wherein R⁵ is a member selected from the group consisting of hydrogen and alkyl.

Compositions are provided comprising any compound of general formula I and a polyglycolysed glyceride, such as an unsaturated polyglycolysed glyceride, a saturated polyglycolysed glyceride, GELUCIRE 33/01, GELUCIRE 35/10, GELUCIRE 37/02, GELUCIRE 44/14, LABRAFIL and LABRASOL.

The composition may further comprise a polyethylene glycol (PEG). Representative polyethylene glycols include, but are not limited to, PEG200, PEG400, PEG600 and PEG2000. A weight ratio of polyglycosylated glyceride to polyethylene glycol (PEG) of from 5:1 to 1:1 is preferred

Also provided, are compositions comprising any compound of general formula I, a polyglycolysed glyceride and peceol (glyceryl monooleate). A weight ratio of polyglycolysed glyceride to peceol of from 9:1 to 1:4 is preferred.

Also provided, are compositions comprising any compound of general formula I, a polyglycolysed glyceride and ethanol. A composition may comprise a compound of general formula I, a polyglycolysed glyceride and 95% ethanol, wherein 95% ethanol and a polyglycolysed glyceride are used in a volume to weight ratio of from 1 to 6.2. Another combination for a composition is a compound of general formula I, GELUCIRE (e.g. GELUCIRE 44/14) and 95% ethanol, wherein 95% ethanol and a polyglycolysed glyceride are used in a volume to weight ratio of from 1 to 6.2 (volume of ethanol to weight of a polyglycolysed glyceride).

The compositions may possess potent antiprogestational activity with minimal antiglucocorticoid activity in combination with improved bioavailability. Therefore, the compositions may be suitable for long term use in the treatment of human endocrinological disorders or other conditions in steroid-sensitive tissues. Specific conditions for treatment include, but are not limited to, endometriosis, dysmenorrhea, uterine leiomyoma, uterine fibroid, meningioma and metastatic breast cancer. Other uses include, but are not limited to, contraception, including emergency post-coital contraception and inducement of cervical ripening.

Also provided is the use of any of the compositions of the present invention in the manufacture of a medicament for treatment of human endocrinological disorders or other conditions in steroid-sensitive tissues as described herein, including but not limited to, endometriosis, dysmenorrhea, uterine leiomyoma, uterine fibroid, meningioma and metastatic breast cancer.

### BRIEF DESCRIPTION OF THE DRAWINGS

FIG. 1. Comparison of bioavailability of CDB4124 in formations comprising GELUCIRE^{R} 44/14 and PEG400 versus formulations comprising GELUCIRE 44/14 and Peceol.

FIG. 2. Comparison of bioavailability of CBD4124 in different formulations comprising GELUCIRE 44/14. G+EtOH50— formulation comprising GELUCIRE 44/14, ethanol and CDB4124 at 50 mg/Kg body weight; G+EtOH200—formulation comprising GELUCIRE 44/14, ethanol and CDB4124 at 200 mg/Kg body weight; G+Pec50—formulation comprising GELUCIRE 44/14, Pecoel and CDB4124 at 50 mg/Kg body weight; G+Pec200—formulation comprising GELUCIRE 44/14, Pecoel and CDB4124 at 200 mg/Kg body weight; G+PEG40—formulation comprising GELUCIRE 44/14, PEG400 and CDB4124 at 40 mg/Kg body weight; G+PEG200—formulation comprising GELUCIRE 44/14, PEG400 and CDB4124 at 200 mg/Kg body weight.

### DETAILED DESCRIPTION OF THE INVENTION

A composition is provided comprising a steroid derivative with the following general formula I:

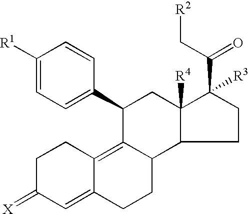

In Formula I, R¹ is a functional group including, but not limited to, —OCH₃, —SCH₃, —N(CH₃)₂, —NHCH₃, —NC₄H₈, —NC₅H₁₀, —NC₄H₈O, —CHO, —CH(OH)CH₃, —C(O)CH₃, —O(CH₂)₂, —N(CH₃)₂, —O(CH₂)₂NC₄H₈, and —O(CH₂)₂NC₅H₁₀. R² is a functional group including, but not limited to, hydrogen, halogen, alkyl, acyl, hydroxy, alkoxy (e.g., methoxy, ethoxy, vinyloxy, ethynyloxy, cyclopropyloxy, etc.), acyloxy (e.g., formyloxy, acetoxy, propionyloxy, heptanoyloxy, glycinate, etc.), alkylcarbonate, cypionyloxy, S-alkyl, —SCN, S-acyl and —OC(O)R⁶, wherein R⁶ is a functional group including, but not limited to, alkyl (e.g., methyl, ethyl, etc.), alkoxyalkyl (e.g., —CH₂OCH₃) and alkoxy (—OCH₃). R¹ is a functional group including, but not limited to, alkyl, hydroxy, alkoxy and acyloxy. R⁴ is a functional group including, but not limited to, hydrogen and alkyl. Finally, X is a functional group including, but not limited to, ═O and ═N—OR⁵, wherein R⁵ is a member selected from the group consisting of hydrogen and alkyl. In a preferred embodiment, R¹, R², R³, R⁴ and X are selected with the proviso that if R¹ is —N(CH₃)₂, R³ is acetoxy, R⁴ is methyl, X is ═O, and R² is not hydrogen.

The term “alkyl” refers to a branched, unbranched, monovalent hydrocarbon radical having from 1-12 carbons. When the alkyl group has from 1-6 carbon atoms, it may be referred to as a “lower alkyl.” Representative alkyl radicals include, for example, methyl, ethyl, n-propyl, i-propyl, 2-propenyl (or allyl), n-butyl, t-butyl, i-butyl (or 2-methylpropyl), etc. As used herein, the term alkyl encompasses “substituted alkyls.” A substituted alkyl refers to alkyl further containing one or more functional groups such as lower alkyl, aryl, aralkyl, acyl, halogen (i.e., alkylhalos, e.g., CF₃), hydroxy (e.g., hydroxymethyl), amino, alkylamino, acylamino, acyloxy, alkoxy (e.g., methoxymethyl), mercapto and the like. These groups may be attached to any carbon atom of the lower alkyl moiety.

The term “alkoxy” may refer to a —OR group, where R is a lower alkyl, substituted lower alkyl, aryl, substituted aryl, aralkyl or substituted aralkyl. Suitable alkoxy radicals include, for example, methoxy, ethoxy, phenoxy, t-butoxy (e.g., methoxyethoxy, methoxymethoxy, etc.), etc.

The term “acyloxy” may refer to an organic radical derived from an organic acid by the removal of a hydrogen. The organic radical can be further substituted with one or more functional groups such as alkyl, aryl, aralkyl, acyl, halogen, amino, thiol, hydroxy, alkoxy, etc. An example of such a substituted organic radical is glycinate (e.g., —OC(O)CH₂NH₂). Suitable acyloxy groups include, for example, acetoxy, i.e., CH₃COO—, which may be derived from acetic acid, formyloxy, i.e., H(CO)O—, which may be derived from formic acid and cypionyloxy, which may be derived from 3-cyclopentylpropionic acid.

The term “halogen” may refer to fluorine, bromine, chlorine and iodine atoms. The term “hydroxyl” may refer to the group —OH. The term “acyl” may denote groups —C(O)R, where R is alkyl or substituted alkyl, aryl or substituted aryl as defined herein. The term “aryl” may refer to an aromatic substituent which may be a single ring or multiple rings which are fused together, linked covalently, or linked to a common group such as an ethylene or methylene moiety. The aromatic ring(s) may include phenyl, naphthyl, biphenyl, diphenylmethyl, 2,2-diphenyl-1-ethyl, and may contain a heteroatom, such as thienyl, pyridyl and quinoxalyl. The aryl group may also be substituted with halogen atoms, or other groups such as nitro, carboxyl, alkoxy, phenoxy, and the like. Additionally, the aryl group may be attached to other moieties at any position on the aryl radical which would otherwise be occupied by a hydrogen atom (such as 2-pyridyl, 3-pyridyl and 4-pyridyl).

The term “alkyl carbonate” may refer to the group —OC(O)OR, where R is alkyl, substituted alkyl, aryl, or substituted aryl as defined herein.

The term “S-alkyl” may refer to the group —SR, where R is lower alkyl or substituted lower alkyl. The term “S-acyl” may refer to a thioester derived from the reaction of a thiol group with an acylating agent. Suitable S-acyls include, for example, S-acetyl, S-propionyl and S-pivaloyl. S-acyl may refer to such thioesters regardless of their method of preparation. The terms “N-oxime” and “N-alkyloxime” may refer to the group ═N—OR⁵, wherein R⁵ is, for example, hydrogen (N-oxime) or alkyl (N-alkyloxime). The oximes can consist of the syn-isomer, the anti-isomer or a mixture of both the syn- and anti-isomers.

Representative compounds within Formula I, include those in which R¹ is —N(CH₃)₂; those in which R² is halogen or alkoxy; those in which R³ is acyloxy; those in which R⁴ is alkyl (e.g., methyl and ethyl); and those is which X is ═O and ═N—OR⁵, wherein R⁵ is hydrogen or alkyl. Additional compounds include those in which R¹ is —N(CH₃)₂; R² is halogen; R³ is acyloxy; and R⁴ is alkyl, such where R² is F, Br or Cl; and R⁴ is methyl. Also included are compounds in which R¹ is —N(CH₃)₂; R² is alkyl; R³ is acyloxy; R⁴ is alkyl; and X is ═O. Also included are compounds in which R¹ is —N(CH₃)₂; R² is alkoxy; R³ is acyloxy; R⁴ is alkyl; and X is ═O. Additional compounds are those in which R² is methoxy or ethoxy; and R³ is acetoxy or methoxy. Also included are compounds in which R¹ is —N(CH₃); R² is hydroxy; R³ is acyloxy; R⁴ is alkyl; and X is ═O. Also included are compounds in which R¹ is —N(CH₃)₂; R² and R³ are both acyloxy; R⁴ is alkyl; and X is ═O. Additional compounds are those in which R² and R³ are both acetoxy. Also included are compounds in which R¹ is —N(CH₃)₂; R² is S-acyl; R³ is hydroxy or acyloxy; R⁴ is alkyl; and X is ═O. Also included are compounds in which R¹ is —N(CH₃)₂; R² is cypionyloxy; R³ is acetoxy; R⁴ is alkyl; and X is ═O. Also included are compounds in which R¹ is —N(CH₃)₂; R² is methoxy; R³ is acetoxy; R⁴ is alkyl; and X is ═O and ═N—OR⁵, wherein R⁵ is, for example, hydrogen or alkyl (e.g., methyl, ethyl, etc.). Also included are compounds in which R¹ is —N(CH₃)₂; R² and R³ are both acetoxy; R⁴ is alkyl; and X is ═O and ═N—OR⁵, wherein R⁵ is, for example, hydrogen or alkyl (e.g., methyl, ethyl, etc.).

Exemplar compounds include, but are not limited to, 17α-acetoxy-11β-[4-(N,N-dimethylamino)phenyl]-21-methoxy-19-norpregna-4,9(10)-diene-3,20-dione with the following structural formula:

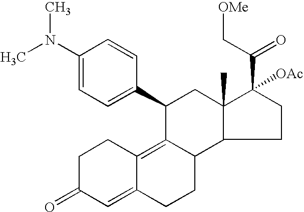

Other exemplar compounds include, but are not limited to, 17α-acetoxy-21-chloro-11-(4-N,N-dimethylaminophenyl)-19-norpregna-4,9-diene-3,20-dione; 17α-acetoxy-21-bromoro-11β-(4-N,N-dimethylaminophenyl)-19-norpregna-4,9-diene-3,20-dione; 17-,21-diacetoxy-11β-(4-N,N-dimethylaminophenyl) 19-norpregna-4,9-diene-3,20-dione; 17α-hydroxy-21-acetylthio-11β-(4-N,N-dimethylaminophenyl)-19-norpregna-4,9-diene-3,20-dione; 17α-acetoxy-21-acetylthio-11β-(4-N,N-dimethylaminophenyl)-19-norpregna-4,9-diene-3,20-dione; 17α-acetoxy-21-ethoxy-11β-(4N,N-dimethylaminophenyl)-19-norpregna-4,9-diene-3,20-dione; 17α-acetoxy-21-methyl-11-(4-N,N-dimethylamino-phenyl)-19-norpregna-4,9-diene-3,20-dione; 17α-acetoxy-21-methoxy-11β-N,N-dimethylaminophenyl)-19-norpregna-4,9-diene-3,20-dione; 17α-acetoxy-21-ethoxy-11β-(4-N,N-dimethylaminophenyl)-19-norpregna-4,9-diene-3,20-dione; 17α-acetoxy-21-(3′-cyclopentylpropionyloxy) 11β-(4-N,N-dimethylaminophenyl)-19-norpregna-4,9-diene-3,20-dione; 17α-acetoxy-21-hydroxy-11β-(4-N,N-dimethylaminophenyl)-19-norpregna-4,9-diene-3,20-dione; 17α,21-diacetoxy-11β-(4-N,N-dimethylaminophenyl) 9-norpregna-4,9-diene-3,20-dione 3-oxime; 17α-acetoxy-21-methoxy-11β-(4-N,N-dimethylaminophenyl)-19-norpregna-4,9-diene-3,2-dione 3-oxime; 17α-acetoxy-1,3-[4-(N-methylamino)phenyl]-19 norpregna-4,9-diene-3,20-dione; and 17α,21-diacetoxy-11β-[4-(N-methylamino)phenyl]-19-norpregna-4,9-diene-3,20-dione.

Also included are those compounds in which R¹ is —N(CH₃)₂, —NC₄H₈, —NC₄H₁₀, —NC₄H₈O, —C(O)CH₃, —O(CH₂)₂N(CH₃)₂, —O(CH₂)₂NC₄H₈, —O(CH₂)₂NC₃H, o, and —O(CH₂)₂NC₅H₁₀; those in which R² is hydrogen, alkyloxy, alkoxy, —SAc, —SCN, —OC(O)CH₂N(CH₃)₂, and —OC(O)R⁶, wherein R⁶ is a functional group including, but not limited to, alkyls (e.g., —CH₂CH₃), alkoxy esters (e.g., —CH₂OMe) and alkoxys (e.g., —OCH₃); those in which R³ is alkyl, alkoxy, acyloxy and hydroxy; those in which R⁴ is alkyl (e.g., methyl and ethyl); and those is which X is ═O or ═N—OR⁵, wherein R⁵ is hydrogen or alkyl. Also preferred are compounds in which R¹ is —N(CH₃)₂; R² is hydrogen; R³ is methoxymethyl; R⁴ is methyl; and X is ═O. Also included are compounds in which R¹ is —N(CH₃)₂; R² is hydrogen; R³ is —OC(O)H, —OC(O)CH₂CH₃ or —OC(O)C₆H₁₃; R⁴ is methyl; and X is ═O. Also included are compounds in which R¹ is —NC₄Hs, —NC₅Ho, —NC₄H₈O, —C(O)CH₃ or —SCH₃; R² is hydrogen; R³ is acetoxy; R⁴ is methyl; and X is ═O. Also included are compounds in which R¹ is —N(CH₃)₂ or —NC₅H₁₀; R² is hydrogen; R³ is methoxy; R⁴ is methyl; and X is ═O. Also included are compounds in which R¹ is —NC₅H₁₀ or —C(O)CH₃; R² and R³ are both acetoxy; R⁴ is methyl; and X is ═O. Also included are compounds in which R¹ is —C(O)CH₃; R² is —SAc; R³ is acetoxy; R⁴ is methyl; and X is ═O. Also included are compounds in which R¹ is —C(O)CH₃, —N(CH₃)₂, —NC₄H; or —NC₅H₁₀; R² and R³ are both methoxy; R⁴ is methyl; and X is ═O. Also included are compounds in which R¹ is —NC₅H₁₀, —C(O)CH₃ or —O(CH₂)₂N(CH₃)₂; R² is methoxy; R³ is acetoxy; R⁴ is methyl; and X is ═O. Also included are compounds in which R¹ is —N(CH₃)₂; R² is —OC(O)CH₂CH₃, —OC(O)OCH₃, —OC(O)OCH₂OCH₃, —OCH═CH₂, —OC(O)CH₂N(CH₃)₂ or —SCN; R³ is acetoxy; R⁴ is methyl; and X is ═O. Also included are compounds in which R¹ is —N(CH₃)₂; R² is —OC(O)H; R³ is —OC(O)H; R⁴ is methyl; and X is ═O. Also included are compounds in which R¹ is —N(CH₃)₂; R² is —OC(O)H; R³ is hydroxy; R⁴ is methyl; and X is ═O. Also included are compounds in which R¹ is —NC₅H₁₀; R² is hydrogen; R³ is acetoxy; R⁴ is methyl; and X is ═N—OR⁵, wherein R⁵ is hydrogen. Also included are compounds in which R¹ is —N(CH₃)₂ or —NC₅H₁₀; R² is hydrogen or methoxy; R³ is methoxy or ethoxy; R⁴ is methyl; and X is ═N—OR⁵, wherein R⁵ is hydrogen.

Exemplar compounds also include, but are not limited to, 17α-acetoxy-11β-[4-(N,N-dimethylamino)phenyl]-21-methoxy-19-norpregna-4,9(10)-diene-3,20-dione; 17α-formyloxy-11β-[4-(N,N-diethylamino)phenyl]-19-norpregna-4,9-diene-3,20-dione; 17α-propionoxy-11β-[4-(N,N-dimethylamino)phenyl]-19-norpregna-4,9-diene-3,20-dione; 17α-heptanoyloxy-11β-[4-(N,N-dimethylamino)phenyl]-19-norpregna-4,9-diene-3,20-dione; 17α-methoxymethyl-11α-[4-N,N-dimethylamino)phenyl]-19-norpregna-4,9-diene-3,20-dione; 17α-acetoxy-11β-(4-N-pyrrolidinophenyl)-19-norpregna-4,9-diene-3,20-dione; 17α-acetoxy-11β-(4-N-piperidinophenyl)-19-norpregna-4,9-diene-3,20-dione; 17α-acetoxy-11β-(4-N-morpholinophenyl)-19-norpregna-4,9-diene-3,20-dione; 17α-acetoxy 11β-(4-acetylphenyl)-19-norpregna-4,9-diene-3,20-dione; 17α-acetoxy-11β-(4-methylthiophenyl)-19-norpregna-4,9-diene-3,20-dione; 17α-methoxy-11β-[4-(N,N-dimethylamino)phenyl]-19-norpregna-4,9-diene-3,20-dione; 17α-methoxy-11β-(4-N-piperidinophenyl)-19-norpregna-4,9-diene-3,20-dione; 17α,21-diacetoxy-11β-(4-N-piperidinophenyl)-19-norpregna-4,9-diene-3,20-dione; 17α,21-diacetoxy-11β-(4 acetylphenyl) 19-norpregna-4,9-diene-3,20-dione; 17α-acetoxy-11β-(4-acetylphenyl)-21-thioacetoxy-19-norpregna-4,9-diene-3,20-dione; 17α,21-dimethoxy-11β-[4-(N,N-dimethylamino)phenyl]-19-norpregna-4,9-diene-3,20-dione; 17α,21-dimethoxy-11β-(4-N-pyrrolidinophenyl)-19-norpregna-4,9-diene-3,20-dione; 17α,21-dimethoxy-11β-(4-N-piperidinophenyl) 19-norpregna-4,9-diene-3,20-dione; 17α,21-dimethoxy-11β-(4-acetylphenyl)-19-norpregna-4,9-diene-3,20-dione; 17α-acetoxy-11β-(4-acetylphenyl)21-methoxy-19-norpregna-4,9-diene-3,20-dione; 17α-acetoxy-11β-{4-[2′-(N,N-dimethylamino)ethoxy]phenyl}-21-methoxy-19-norpregna-4,9-diene-3,20-dione; 17α,21-diformyloxy-11β-[4-(N,N-dimethylamino)phenyl]-19-norpregna-4,9-diene-3,20-dione; 17α-acetoxy-11β-[4-(N,N-dimethylamino)phenyl]-21-propionyloxy-19-norpregna-4,9-diene-3,20-dione; 17α-acetoxy-11β-[4-(N,N-dimethylamino)phenyl]-21-(2′-methoxyacetyl)oxy-19-norpregna-4,9-diene-3,20-dione; 17α-acetoxy-21-hydroxy-11β-[4-(N,N-dimethylamino)phenyl]-19-norpregna-4,9-diene-3,20-dione-21-methyl carbonate; 17α-acetoxy-11β-[4-(N,N-dimethylamino)phenyl]-21-(1′-ethenyloxy)-19-norpregna-4,9-diene-3,20-dione; 17α-acetoxy-11β-[4-(N,N-dimethylamino)phenyl]-21-(2′-N,N-dimethylamino)acetoxy-19-norpregna-4,9-diene-3,20-dione; 17α-acetoxy-11β-[4-(N,N-dimethylamino)phenyl]-21-thiocyanato-19-norpregna-4,9-diene-3,20-dione; 17α-acetoxy-11β-(4-N-piperidinophenyl)-19-norpregna-4,9-diene-3,20-dione 3-oxime; 17α-methoxy-11β-[4-(N,N-dimethylamino)phenyl]-19-norpregna-4,9-diene-3,20-dione 3-oxime; 17α-methoxy-11β-(4-N-piperidinophenyl)-19-norpregna-4,9-diene-3,20-dione 3-oxime; and 17α,21-dimethoxy-11β-[4-(N,N-dimethylamino)phenyl]-19-norpregna-4,9-diene-3,20-dione 3-oxime.

The absorption of a compound having general formula I into the bloodstream of a mammal may be significantly improved when the compound is administered to the mammal as a formulation with a polyglycolysed glyceride. A composition is provided comprising a compound of formula I, a polyglycolysed glyceride and optionally a pharmaceutically acceptable carrier. The composition may further comprise a polyethylene glycol (PEG), such as a polyethylene glycol with molecular weight in a range from 200 to 35000 or such as PEG with molecular weight 400 (PEG400). Alternatively, the composition may further comprise ethanol or Peceol.

“Polyglycolysed glycerides” may be a mixture of mono-, di- and triglycerides and polyethylene glycol (PEG) mono- and diesters, which may be of molecular weight between 200 and 600, where appropriate of free glycerol and free PEG, whose HLB (Hydrophile-Lipophile Balance) value may be adjusted by the length of the PEG chain, and whose melting point is adjusted by the length of the chains of the fatty acids, of the PEG and by the degree of saturation of the fatty chains, and hence of the starting oil; examples of such mixtures are GELUCIRE. Another suitable saturated polyglyocylsed glyceride is LABRASOL, a mixture of polyoxyethylene glyceryl caprylate and polyoxyethylene glyceryl caproate.

GELUCIRE compositions may be inert semi-solid waxy materials which are amphiphilic in character and are available with varying physical characteristics. They are surface active in nature and disperse or solubilize in aqueous media forming micelles, microscopic globules or vesicles. They are identified by their melting point/HLB value. The melting point is expressed in degrees Celsius and the HLB is a numerical scale extending from 0 to approximately 20. Lower HLB values denote more lipophilic and hydrophobic substances, and higher values denote more hydrophilic and lipophobic substances. The affinity of a compound for water or for oily substances is determined and its HLB value is assigned experimentally. One or a mixture of different grades of GELUCIRE excipient may be chosen to achieve the desired characteristics of melting point and/or HLB value. GELUCIRE compositions are mixtures of monoesters, diesters and/or triesters of glycerides of long chain (C₁₂ to C₁₈) fatty acids, and PEG (mono- and/or di) esters of long chain (C₁₂ to C₁₈) fatty acids and can include free PEG. GELUCIRE compositions are generally described as fatty acid esters of glycerol and PEG esters or as polyglycolysed glycerides. GELUCIRE compositions are characterized by a wide range of melting points of from about 33° C. to about 64° C. and most commonly from about 35° C. to about 55° C., and by a variety of HLB values of from about 1 to about 14, most commonly from about 7 to about 14. For example, GELUCIRE 44/14 designates a melting point of approximately 44° C. and an HLB value of about 14 to this grade of GELUCIRE.

A polyglycolysed glyceride may be saturated or unsaturated. Saturated polyglycolysed glycerides are obtainable by partial alcoholysis of hydrogenated vegetable oil with polyethylene glycol or by esterification of saturated fatty acids with polyethylene glycol and glycerol. Unsaturated polyglycolysed glycerides may be obtained by partial alcoholysis of non-hydrogenated vegetable oil with polyethylene glycol.

Saturated polyglycolysed glycerides include those comprising C₈-C₁₈ glycerides and polyethylene glycol esters, such as GELUCIRE 33/01, 35/10, 37/02 or 44/14 and LABRAFIL WL 2514 CS as well as those comprising C₈-C₁₀ glycerides and polyethylene glycol esters, such as those available under the trade name LABRASOL. Unsaturated polyglycolysed glycerides include apricot kernel oil PEG-6 complex (LABRAFIL M-1944 CS), almond oil PEG-6 complex (LABRAFIL M-1966 CS), peanut oil PEG-6 complex (LABRAFIL M-1969 CS), olive oil PEG-6 complex (LABRAFIL M-1980 CS), corn oil PEG-6 complex (LABRAFIL M-2125 CS) and LABRAFIL WL 2609 BS. A mixture of polyglycolysed glycerides may also be employed, such as GELUCIRE 44/14 and LABRASOL.

A polyglycolysed glyceride may comprise 5 to 100%, or 20 to 80% by weight of the total excipients. Thus, the polyglycolysed glyceride may be used as the sole carrier for a compound of formula I, or may be admixed with other excipients.

The polyglycolysed glyceride may be used in combination with a polyethylene glycol. A weight ratio of polyglycolysed glyceride to polyethylene glycol (PEG) of from 5:1 to 1:1 is preferred. For example, the polyglycolysed glyceride and the polyethylene glycol can be used in the range of the following ratios of a polyglycolysed glyceride to PEG: 5:1, 4:1, 3:1, 2:1 or 1:1. The composition may comprise 2.87 parts of a polyglycolysed glyceride to 1 part of a polyethylene glycol, such as PEG400.

In addition, a polyglycolysed glyceride may be used in combination with peceol. Peceol is a readily dispersible, solubilizing agent comprised primarily of a mixture of mono- and diglycerides of oleic acid that closely resembles the end products of intestinal lipid digestion (Hauss et al. 1998. J. Pharm. Sci. 87:164-169). Previous studies have demonstrated a significant increase in the absorption of the hydrophobic drug cyclosporine from predigested olive oil, when compared to that of a nondigested control (Porter et al. 2001. Adv. Drug Delivery Rev. 50:61-80).

A weight ratio of polyglycolysed glyceride to peceol of from 9:1 to 1:4 is preferred. For example, polyglycolysed glylceride and peceol can be used in the following ratios: 90% of a polyglycolysed glylceride to 10% of peceol, 80% of a polyglycolysed glylceride to 20% of peceol, 70% of a polyglycolysed glylceride to 30% of peceol, 60% of a polyglycolysed glylceride to 40% of peceol, 55% of a polyglycolysed glylceride to 45% of peceol, 50% of a polyglycolysed glylceride to 50% of peceol, 45% of a polyglycolysed glylceride to 55% of peceol, 40% of a polyglycolysed glylceride to 60% of peceol, 30% of a polyglycolysed glylceride to 70% of peceol, 20% of a polyglycolysed glylceride to 80% of peceol. The combination may be 50% GELUCIRE 44/14 to 50% part of peceol comprising glyceryl monooleate.

A polyglycolysed glyceride can also be used in a combination with 95% ethanol. The ratio between 95% ethanol and the polyglycolysed glyceride may be 1 to 6.2 (volume of ethanol to weight of a polyglycolysed glyceride) such as 95% ethanol and GELUCIRE at ratio 1 to 6.2 (volume of ethanol to weight of a polyglycolysed glyceride).

The compositions may possess potent antiprogestational activity and minimal antiglucocorticoid activity, combined with improved absorption, which may render these compositions suitable for oral administration.

The compositions can be advantageously used, inter alia, to antagonize endogenous progesterone; to induce menses; to treat endometriosis; to treat dysmenorrhea; to treat endocrine hormone-dependent tumors; to treat meningioma; to treat uterine leiomyonas, to treat uterine fibroids; to inhibit uterine endometrial proliferation; to induce labor; to induce cervical ripening, for hormone therapy; and for contraception.

The compositions having antiprogestational activity may also be characterized by antagonizing the effects of progesterone. As such, the compositions may be of particular value in the control of hormonal irregularities in the menstrual cycle, for controlling endometriosis and dysmenorrhea, and for inducing menses. In addition, the compositions can be used as a method of providing hormone therapy either alone or in combination with estrogenic substances in postmenopausal women, or in women whose ovarian hormone production is otherwise compromised.

Moreover, the compositions can be used for control of fertility during the whole of the reproductive cycle. For long-term contraception, the compositions can be administered either continuously or periodically depending on the dose. In addition, the compositions may be of particular value as post-coital contraceptives, for rendering the uterus inimical to implantation, and as “once a month” contraceptive agents.

A further important utility for the compositions lies in their ability to slow down growth of hormone-dependent tumors and/or tumors present in hormone-responsive tissues. Such tumors include, but are not limited to, kidney, breast, endometrial, ovarian, and prostate tumors, e.g., cancers, which may be characterized by possessing progesterone receptors. In addition, such tumors include meningiomas. Other utilities of the compositions include the treatment of fibrocystic disease of the breast and uterine.

The compositions can be administered to any warm-blooded mammal such as humans, domestic pets, and farm animals. Domestic pets include dogs, cats, etc. Farm animals include cows, horses, pigs, sheep goats, etc.

The amount of active ingredient that can be combined with an optimal carrier material to produce a single dosage form will vary depending upon the disease treated, the mammalian species, and the particular mode of administration. For example, a unit dose may comprise between 0.1 milligram and 1 gram of the active ingredient or between 0.001 and 0.5 grams. However, the specific dose level for any particular patient will depend on a variety of factors including the activity of the specific compound employed; the age, body weight, general health, sex and diet of the individual being treated; the time and route of administration; the rate of excretion; other drugs which have previously been administered; and the severity of the particular disease undergoing therapy.

The compositions can be administered by a variety of methods. For example, the compositions can be administered via the oral route in a form of solutions, suspensions, emulsions, tablets, including sublingual and intrabuccal tablets, soft gelatin capsules, including solutions used in soft gelatin capsules, aqueous or oil suspensions, emulsions, pills, lozenges, troches, tablets, syrups or elixirs and the like.

The compositions can be also administered as an implant including SILASTIC and biodegradable implants or via intramuscular and intravenous injections.

The compositions can contain one or more agents selected from the group consisting of sweetening agents, flavoring agents, coloring agents and preserving agents. Tablets containing the active ingredient in admixture with nontoxic pharmaceutically acceptable excipients, which are suitable for manufacture of tablets are acceptable. These excipients can be, for example, inert diluents, such as calcium carbonate, sodium carbonate, lactose, calcium phosphate or sodium phosphate, granulating and disintegrating agents, such as maize starch, or alginic acid; binding agents, such as starch, gelatin or acacia; and lubricating agents, such as magnesium stearate, stearic acid and talc. Tablets can be uncoated or, alternatively, they can be coated by known methods to delay disintegration and adsorption in the gastrointestinal tract and thereby provide a sustained action over a longer period. For example, a time delay such as glyceryl monostearate or glyceryl distearate alone or with a wax can be employed.

Formulations for oral use can also be presented as hard gelatin capsules wherein the active ingredient is mixed with an inert solid diluent, for example calcium carbonate, calcium phosphate or kaolin, or as soft gelatin capsules wherein the active ingredient is mixed with water or an oil medium, such as peanut oil, liquid paraffin or olive oil.

Aqueous suspensions may contain the active materials in admixture with excipients suitable for the manufacture of aqueous suspensions. Such excipients include a suspending agent, such as sodium carboxymethylcellulose, methylcellulose, hydroxypropylmethylcellulose, sodium alginate, polyvinylpyrrolidone, gum tragacanth and gum acacia, and dispersing or wetting agents such as a naturally occurring phosphatide (e.g., lecithin), a condensation product of an alkylene oxide with a fatty acid (erg., polyoxyethylene stearate), a condensation product of ethylene oxide with a long chain aliphatic alcohol (e.g., heptadecaethylene oxycetanol), a condensation product of ethylene oxide with a partial ester derived from a fatty acid and a hexitol (e.g., polyoxyethylene sorbitol mono-oleate), or a condensation product of ethylene oxide with a partial ester derived from fatty acid and a hexitol anhydride (e.g. polyoxyethylene sorbitan monooleate). The aqueous suspension can also contain one or more preservatives such as ethyl or n-propyl p-hydroxybenzoate, one or more coloring agents, one or more flavoring agents and one or more sweetening agents, such as sucrose, aspartame or saccharin.

Oil suspensions can be formulated by suspending the active ingredient in a vegetable oil, such as arachis oil, olive oil, sesame oil or coconut oil, or in a mineral oil such as liquid paraffin. The oil suspensions can contain a thickening agent, such as beeswax, hard paraffin or cetyl alcohol. Sweetening agents can be added to provide a palatable oral preparation. These compositions can be preserved by the addition of an antioxidant such as ascorbic acid.

Dispersible powders and granules suitable for preparation of an aqueous suspension by the addition of water can be formulated from the active ingredients in admixture with a dispersing, suspending and/or wetting agent, and one or more preservatives. Suitable dispersing or wetting agents and suspending agents are exemplified by those disclosed above. Additional excipients, for example sweetening, flavoring and coloring agents, can also be present.

The pharmaceutical composition can also be in the form of oil-in-water emulsions. The oily phase can be a vegetable oil, such as olive oil or arachis oil, a mineral oil, such as liquid paraffin, or a mixture of these. Suitable emulsifying agents include naturally-occurring gums, such as gum acacia and gum tragacanth, naturally occurring phosphatides, such as soybean lecithin, esters or partial esters derived from fatty acids and hexitol anhydrides, such as sorbitan monooleate, and condensation products of these partial esters with ethylene oxide, such as polyoxyethylene sorbitan monooleate. The emulsion can also contain sweetening and flavoring agents.

Syrups and elixirs can be formulated with sweetening agents, such as glycerol, sorbitol or sucrose. Such formulations can also contain a demulcent, a preservative, a flavoring or a coloring agent.

The pharmaceutical composition can be in the form of a sterile injectable preparation, such as a sterile injectable aqueous or oleaginous suspension. This suspension can be formulated using those suitable dispersing or wetting agents and suspending agents which have been mentioned above. The sterile injectable preparation can also be a sterile injectable solution or suspension in a nontoxic parenterally-acceptable diluent or solvent, such as a solution of 1,3-butanediol. Among the acceptable vehicles and solvents that can be employed are water and Ringer's solution, an isotonic sodium chloride. In addition, sterile fixed oils can be employed as a solvent or suspending medium. For this purpose any bland fixed oil can be employed including synthetic mono- or diglycerides. In addition, fatty acids such as oleic acid can likewise be used in the preparation of injectables.

The compound can also be administered in the form of suppositories for rectal administration of the drug. These compositions can be prepared by mixing the drug with a suitable non-irritating excipient which is solid at ordinary temperatures but liquid at the rectal temperatures and will therefore melt in the rectum to release the drug. Such materials are cocoa butter and polyethylene glycols.

They can also be administered by in intranasal, intraocular, intravaginal, and intrarectal routes including suppositories, insufflation, powders and aerosol formulations.

Compounds administered by the topical route can be administered as applicator sticks, solutions, suspensions, emulsions, gels, creams, ointments, pastes, jellies, paints, powders, and aerosols.

The invention will be described in greater detail by way of the following specific, but not limiting, examples.

### EXAMPLE 1

### Preparing a Semi-Solid Composition Comprising GELUCIRE 44/14, PEG400 and 17α-acetoxy-11β-[4-(N,N-dimethylamino)phenyl]-21-methoxy-19-norpregna-4,9(10)-diene-3,20-dione

GELUCIRE 44/14 (lauroyl macrogol-32 glycerides) and PEG400 (polyethylene glycol 400) were mixed in a beaker in the weight ratio 74.1% GELUCIRE 44/14 and 25.9% PEG400. The mixture was heated to 50 to 54° C. until GELUCIRE 44/14 was melted to completion. The mixture was held at the temperature of 48-50° C. for 10 to 15 minutes until the mixture became a clear solution. 17α-acetoxy-11β-[4-(N,N-dimethylamino)phenyl]-21-methoxy-19-norpregna-4,9(10)-diene-3,20-dione was then added to the mixture to the final concentration of 3.43% mixture weight (35 mg/ml). After 17α-acetoxy-11β-[4-(N,N-dimethylamino)phenyl]-21-methoxy-19-norpregna-4,9(10)-diene-3,20-dione was dissolved to completion, the solution was held at 48 to 52° C. with good mixing for additional 10 to 15 minutes. The solution was then transferred to a suitable container.

### EXAMPLE 2

### Preparing a Composition Comprising GELUCIRE 44/14, PEG400 and 17α-acetoxy-11β-[4-(N,N-dimethylamino)phenyl]-21-methoxy-19-norpregna-4,9(10)-diene-3,20-dione in a Capsule Form

PEG400 was heated to 50° C., GELUCIRE 44/14 was then added in the 2.87:1 ratio of GELUCIRE 44/14 to PEG400. The mixture was then heated with good mixing until GELUCIRE 44/14 was melted to completion. 17α-acetoxy-11β-[4-(N,N-dimethylamino)phenyl]-21-methoxy-19-norpregna-4,9(10)-diene-3,20-dione was then added to the concentration of 3.42%. The solution was then held at 50° C. for 30 minutes. Empty pre-weighted capsules were then filled with the solution at the target weight of net fill of 365 mg per capsule.

### EXAMPLE 3

### Preparing a Semi-Solid Composition Comprising GELUCIRE 44/14, Peceol and 17α-acetoxy-11β-[4-(N,N-dimethylamino)phenyl]-21-methoxy-19-norpregna-4,9(10)-diene-3,20-dione

GELUCIRE 44/14 (lauroyl macrogol-32 glycerides EP) and Peceol (glyceryl monooleate 40) were mixed in a beaker in the weight ratio 50% GELUCIRE 44/14 and 50% Peceol. The mixture was heated to 50 to 54° C. until GELUCIRE 44/14 was melted to completion. The mixture was held at the temperature of 48-50° C. for 10 to 15 minutes until the mixture became a clear solution. 17α-acetoxy-11β-[4-(N,N-dimethylamino)phenyl]-21-methoxy-19-norpregna-4,9(10)-diene-3,20-dione was then added to the mixture to the final concentration of 3.43% mixture weight. After 17α-acetoxy-11β-[4-(N,N-dimethylamino)phenyl]-21-methoxy-19-norpregna-4,9(10)-diene-3,20-dione was dissolved to completion, the solution was held at 48 to 52° C. with good mixing for additional 10 to 15 minutes. The solution was then transferred to a suitable container.

### EXAMPLE 4

### Preparing a Composition Comprising GELUCIRE 44/14, Peceol and 17α-acetoxy-11β-[4-(N,N-dimethylamino)phenyl]-21-methoxy-19-norpregna-4,9(10)-diene-3,20-dione in a Capsule Form

A solution of 17α-acetoxy-11β-[4-(N,N-dimethylamino)phenyl]-21-methoxy-19-norpregna-4,9(110)-diene-3,20-dione, GELUCIRE, and Pecoel was prepared as described in Example 2, except the final concentration of 17α-acetoxy-11β-[4-(N,N-dimethylamino)phenyl]-21-methoxy-19-norpregna-4,9(10)-diene-3,20-dione was 4.34%. The solution was then added to pre-weighted empty capsules at 28 μl per one capsule. Upon filling, capsules were re-weighted and the capsules with 0.28-0.29 g of net weight were selected for being used in treatment protocols. Final concentration of 17α-acetoxy-11β-[4-(N,N-dimethylamino)phenyl]-21-methoxy-19-norpregna-4,9(10)-diene-3,20-dione per capsule was 12.2-12.6 mg.

### EXAMPLE 5

### Preparing a Semi-Solid Composition Comprising GELUCIRE 44/14, Ethanol and 17α-acetoxy-11β-[4-(N,N-dimethylamino)phenyl]-21-methoxy-19-norpregna-4,9(10)-diene-3,20-dione

17α-acetoxy-11β-[4-(N,N-dimethylamino)phenyl]-21-methoxy-19-norpregna-4,9(10)-diene-3,20-dione was dissolved in 95% ethanol by ratio of one to one (weight to volume. GELUCIRE 44/14 was melted by heating the GELUCIRE 44/14 powder with constant mixing. A solution of 17α-acetoxy-11β-[4-(N,N-dimethylamino)phenyl]-21-methoxy-19-norpregna-4,9(10)-diene-3,20-dione in 95% ethanol was then added to melted GELUCIRE 44/14 with the ratio of 1 to 6.2 (volume to weight; 1 ml to 6.2 gram). The final concentration of 17α-acetoxy-11β-[4-(N,N-dimethylamino)phenyl]-21-methoxy-19-norpregna-4,9(10)-diene-3,20-dione was 12.5% (w/w).

### EXAMPLE 6

### Bioavailability of Various Compositions Comprising GELUCIRE 44/14 in Dogs

Twelve adult female Beagle dogs with a body weight in the range of 8.5 to 10.7 kg were divided into two groups: I, and II. This was a single dose study. As shown in Table 1 below, dogs in group I received capsules with 12.5 mg of 17α-acetoxy-11β-[4-(N,N-dimethylamino)phenyl]-21-methoxy-19-norpregna-4,9(10)-diene-3,20-dione per capsule (listed under laboratory log name CDB 4124) in formulation with GELUCIRE and Pecoel, while dogs in group II received capsules with the same amount of 17α-acetoxy-11β-[4-(N,N-dimethylamino)phenyl]-21-methoxy-19-norpregna-4,9(10)-diene-3,20-dione (12.5 mg per dose), but in formulation with GELUCIRE and PEG400.

TABLE 1

| | | # of | |
|---|---|---|---|
| Group | Formulation | capsule | CDB4124 |
| I | CDB4124 + GELUCIRE + Pecoel | 1 | 12.5 mg |
| II | CDB4124 + GELUCIRE + PEG400 | 1 | 12.5 mg |

Blood was drawn at 0.25, 0.5, 1, 2, 4, 8, 16, and 24 hours ±5 minutes following dosing. The plasma samples were collected and analyzed for presence of 17α-acetoxy-11β-[4-(N,N-dimethylamino)phenyl]-21-methoxy-19-norpregna-4,9(10)-diene-3,20-dione (under laboratory log name CDB4124).

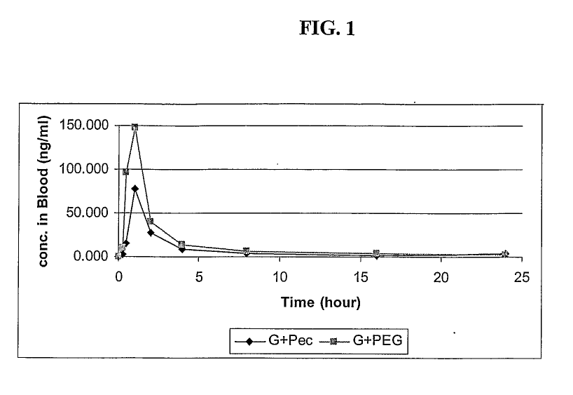

The results of these tests are shown in FIG. 1 as concentration of CDB4124 level in blood (ng/ml) versus time (in hours) after dosing. The maximum concentration achieved for CDB4124 formulations with GELUCIRE and PEG400 was approximately two hours after dosing. Area under Curve (AUC) measurements by weight were undertaken and results of these measurements are presented in Table 2.

TABLE 2
AUC by weight

| | | Ratio to | Ratio to |
|---|---|---|---|
| | Wt.(mg) | G + Pec | G + PEG |
| G + Pec | 81.1 | 1.0 | 0.5 |
| G + PEG | 157.83 | 1.9 | 1.0 |

### EXAMPLE 7

### Bioavailability of Various Compositions Comprising GELUCIRE 44/14 in Rats

To examine bioavailability of CDB4124 compositions comprising GELUCIRE 44/14 and Ethanol, thirty female rats were randomly assigned to one of four Groups I, II, III or IV, with nine rats in Groups I, II, III, and 3 rats in Group IV. Group IV was a control group. After an overnight fast, each rat in Group I, II, and III was dosed with the test article by oral gavage according to the group assignment. Group I animals were dosed at 12.5 mg/kg body weight of CDB4124, Group II animals were dosed at 50 mg/kg body weight of CDB4124, and Group III animals were dosed at 200 mg/kg body weight of CDB4124. Blood samples were drawn at 0.5, 1, 4, 6, 12 and 24 hours after dosing and on Day 11. The levels of CDB4124 in the blood at the various time points were measured.

To examine bioavailability of CDB4124 compositions comprising GELUCIRE 44/14 and Peceol, twenty female rats were used for the study. Four groups, I, II, III, or IV with five rats in each group were assigned to 0 mg/kg body weight of CDB4124, 10 mg/kg body weight of CDB4124, 40 mg/kg body weight of CDB4124, and 200 mg/kg body weight of CDB4124, respectively. After an overnight fast, each rat in Group I, II, III, and IV was dosed with the test article by oral gavage according to the group assignment. Blood samples were drawn at 0, 1, 2, 4, 8, 12, and 24 hours after dosing and on Day 14. The blood samples at different time points were analyzed for the concentration of CDB4124.

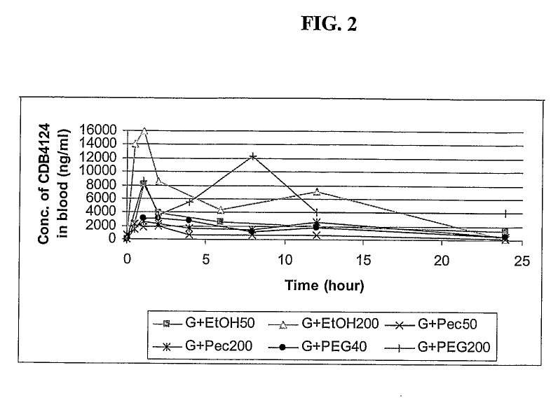

To examine bioavailability of CDB4124 compositions comprising GELUCIRE 44/14 and PEG400, one hundred and seventy rats were used for the study. The rats were divided into four groups, Group I: control (35 rats, 0 mg/kg body weight), Group II: 45 rats (10 mg/kg body weight), Group III: 45 rats (40 mg/kg body weight), and Group IV: 45 rats (200 mg/kg body weight). Nine rats from each group except control group were assigned for blood draw at different time points (two time points/rat). The time points included 1, 2, 4, 8, 12, and 24 hours after the first dosing. The blood samples were analyzed for the concentration of CDB4124. The results of these measurements are presented in FIG. 2 as concentration of CBD4124 in blood (ng/ml) at different time points post dosing for each of the tested formulations. Results for the following formulations were plotted in FIG. 2: 1) formulation comprising GELUCIRE 44/14, ethanol and CDB4124 at 50 mg/kg body weight (G+EtOH50); 2) formulation comprising GELUCIRE 44/14, ethanol and CDB4124 at 200 mg/kg body weight (G+EtOH200); formulation comprising GELUCIRE 44/14; 3) formulation comprising GELUCIRE^{R} 44/14, Pecoel and CDB4124 at 50 mg/kg body weight (G+Pec50); 4) formulation comprising GELUCIRE 44/14, Pecoel and CDB4124 at 200 mg/kg body weight (G+Pec200); 5) formulation comprising GELUCIRE 44/14, PEG400 and CDB4124 at 40 mg/kg body weight (G+PEG40); 6) formulation comprising GELUCIRE 44/14, PEG400 and CDB4124 at 200 mg/kg body weight (G+PEG200).

Table 3 provides data on CDB4124 measurements at different time points for different formulations.

TABLE 3
CDB-4124 in blood (ng/ml)

| (Hour) | G + EtOH | G + EtOH | G + Pec* | G + Pec* | G + PEG | G + PEG |
|---|---|---|---|---|---|---|
| Time 0 | 50 mg/kg | 200 mg/kg | 50 mg/kg | 200 mg/kg | 40 mg/kg | 200 mg/kg |
| 0 | 0 | 0 | 29.4 | 678 | 0 | 0 |
| 0.5 | 2273 | 14110 | 2221.5 | 1471 | | |
| 1 | 8119 | 15994 | 1799 | 2648.3 | 3129.3 | 8521.7 |
| 2 | 3956 | 8643 | 1886.7 | 2201.3 | 3056 | 3496.5 |
| 4 | | | 639.7 | 1693 | 2713.7 | 5511 |
| 6 | 2656 | 4298 | | | | |
| 8 | | | 692.7 | 1408.3 | 1077.7 | 12295 |
| 12 | 1966 | 7182 | 651.7 | 2615.3 | 1754.7 | 4068.7 |
| 24 | 1216 | 88 | 176.9 | 463.5 | 409 | 3965 |

The ratios of AUC (Area under Curve) of individual treatments to that of other treatments are listed in the Table 4.

TABLE 4
AUC by paper weight and ratios for different treatments.

| | Paper | Ratio to | Ratio to | Ratio to |
|---|---|---|---|---|
| | Wt.(mg) | G + PEG40 | G + EtOH | G + Pec50 |
| G + PEG40 | 119.4 | 1.0 | 0.7 | 2.0 |
| G + EtOH50 | 176.52 | 1.5 | 1.0 | 3.0 |
| G + Pec50 | 58.3 | 0.5 | 0.3 | 1.0 |

1. A composition comprising a polyglycolysed glyceride and a compound having the general formula: 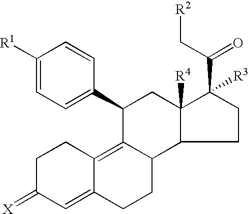
wherein:
R¹ is a member selected from the group consisting of —N(CH₃)₂, —NHCH₃, —NC₄H₈, —NC₅H₁₀, and —NC₄H₈O;
R² is a member selected from the group consisting of hydrogen, halogen, alkyl, acyl, hydroxy, alkoxy, acyloxy, alkylcarbonate, cypionyloxy, S-alkyl, —SCN, S-acyl, and —OC(O)R⁶ wherein R⁶ is a member selected from the group consisting of alkyl, alkoxy ester and alkoxy;
R³ is a member selected from the group consisting of alkyl, hydroxy, alkoxy and acyloxy;
R⁴ is a member selected from the group consisting of hydrogen and alkyl; and
X is a member selected from the group consisting of ═O and ═N—OR⁵, wherein R⁵ is a member selected from the group consisting of hydrogen and alkyl.

2. The composition of claim 1, wherein R¹ of said compound is a member selected from the group consisting of —N(CH₃)₂, and —NHCH₃.

3-44. (canceled)

45. The composition of claim 1, wherein said compound is 17α-acetoxy-11β-[4-(N,N-dimethylamino)phenyl]-21-methoxy-19-norpregna-4,9(10)-diene-3,20-dione.

46. The composition of claim 1 wherein the compound has the structural formula:

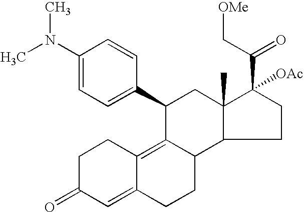

47. The composition of claim 1, wherein the polyglycolysed glyceride consists of C₈-C₁₈ glycerides and polyethylene glycol esters.

48. The composition of claim 1, wherein the polyglycolysed glyceride is GELUCIRE 44/14.

49. The composition of claim 1, wherein the polyglycolysed glyceride is selected from the group consisting: GELUCIRE 35/10, 37/02, 44/14, 50/13, WL 2514CS and LABRASOL.

50. The composition of claim 1 or 46 further comprising polyethylene glycol (PEG).

51. The composition of claim 50 wherein the polyethylene glycol is PEG400.

52. The composition of claim 51 wherein the polyglycolysed glyceride is GELUCIRE 44/14.

53. The composition of claim 1 or 46, further comprising ethanol.

54. The composition of claim 53 wherein the ethanol is 95% Ethanol.

55. The composition of claim 1 or 46, further comprising peceol.

56. The composition of claim 55 wherein the polyglycolysed glyceride is GELUCIRE 44/14.

57. A method of producing an antiprogestational effect in a patient, said method comprising administering to said patient an effective amount of the composition of claim 1 or 46.

58. A method of inducing menses in a patient, said method comprising administering to said patient an effective amount of the composition of claim 1 or 46.

59. A method of treating endometriosis, said method comprising administering to said patient an effective amount of the composition of claim 1 or 46.

60. A method of treating dysmenorrhea, said method comprising administering to said patient an effective amount of the composition of claim 1 or 46.

61. A method of treating endocrine hormone-dependent tumors, said method comprising administering to said patient an effective amount of the composition of claim 1 or 46.

62. A method of treating meningiomas, said method comprising administering to said patient an effective amount of the composition of claim 1 or 46.

63. A method of treating uterine fibroids in a patient, said method comprising administering to said patient an effective amount of the composition of claim 1 or 46.

64. A method of inhibiting uterine endometrial proliferation in a patient, said method comprising administering to said patient an effective amount of the composition of claim 1 or 46.

65. A method of inducing labor, said method comprising administering to a patient an effective amount of the composition of claim 1 or 46.

66. A method of contraception, said method comprising administering to a patient an effective amount of the composition of claim 1 or 46.

67. A method of post-coital contraception, said method comprising administering to a patient an effective amount of the composition of claim 1 or 46.
